(19) United States
(12) Patent Application Publication
(10) Pub. No.: US 2002/0137060 A1
(43) Pub. Date: Sep. 26, 2002

(54) Combined hybridization-detection assays for determining nucleic acid concentrations in biological fluids

(76) Inventors: Patricia Lea Brown-Augsburger, Fishers, IN (US); Xin Min Yue, Carmel, IN (US); James Anthony McSwiggen, Boulder, CO (US)

Correspondence Address: ELI LILLY AND COMPANY, Patent Division/CEC, Lilly Corporate Center, Indianapolis, IN, 46285, US

(21) Appl. No.: 09/927,014

(22) Filed: Aug. 9, 2001

Related U.S. Application Data
(60) Provisional application No. 60/224,164, filed on Aug. 9, 2000.

(51) Int. Cl. C12Q 1/68

(52) U.S. Cl. 435/6

(57) ABSTRACT

Provided are combined nucleic acid hybridization-detection assays for detecting, or determining the concentration of, a target nucleic acid in a sample of biological fluid; detecting, or determining the concentration of, each one of two or more target nucleic acids in a sample of biological fluid; determining the amount of a metabolite of a target nucleic acid in a biological fluid; determining the amounts of multiple metabolites of a target nucleic acid in a biological fluid; detecting a metabolite of a target nucleic acid in a biological fluid; detecting multiple metabolites of a target nucleic acid in a biological fluid; and for determining the concentration of a ribozyme in mammalian serum.

This application claims the benefit of priority of U.S. Provisional Patent Application Serial No. 60/224,164 filed Aug. 9, 2000, the contents of which are herein incorporated by reference in their entirety.

### BACKGROUND OF THE INVENTION

1. Field of the Invention

The present invention relates to the field of assays. More particularly, the present invention relates to the field of diagnostic assays, in particular combined hybridization-detection assays for determining the concentration of nucleic acids, especially therapeutically useful nucleic acids such as ribozymes and antisense oligonucleotides, in body fluids such as blood, serum, urine, saliva, etc. Such assays are useful for pharmacokinetic and metabolic studies during drug development, toxicological and clinical studies, and in optimizing the therapeutic regimen of patients undergoing treatment for conditions responsive to such nucleic acids. These assays are useful in both human and veterinary medicine.

2. Description of Related Art

Ribozymes are polymers of natural or modified ribonucleotides that are capable of catalyzing the cleavage of a complementary strand of RNA. These molecules are currently being developed for human therapeutic use (Kruger et al. (1999) Methods in Enzymology 306:207-25; Muotri et al. (1999) Gene 237:303-10). Antisense oligonucleotides represent another class of nucleotide polymers also being developed for therapeutic applications (Gewirtz et al. (1998) Blood 92(3):712-36; Galderisi et al. (1999) Journal of Cellular Physiology 181(2):251-7). Both types of molecules are designed to stop transcription and/or translation of an RNA message. In addition, antisense oligonucleotides have also been shown to interfere with the activity of gene products. As molecules like ribozymes and antisense oligonucleotides (referred to hereinafter collectively as “therapeutic nucleic acids”) are developed as drugs, the need arises to detect the presence of, and/or measure the concentrations of, these compounds in blood and other biological matrices in order to optimize drug design, and monitor and optimize patient treatment regimens.

Conventional approaches to bioanalytical quantification of nucleic acids have included HPLC, electrophoresis, or capillary gel electrophoresis. Overall, these approaches have limited sensitivity (50 to 100 ng/mL), and are time intensive because they require extraction of the nucleic acid of interest from a biological matrix. This extraction step may contribute to limited assay sensitivity due to incomplete recovery of the analyte.

Previous bioanalytical methodology for determining ribozyme or antisense oligonucleotide concentrations in biological/physiological fluids has required extraction of the drug from the biological matrix, followed by chromatography, electrophoresis, or capillary gel electrophoresis for quantitation (Leeds et al. (1997) Drug Metabolism and Disposition 25(8):921-926; Desjardins et al. (1996) Journal of Pharmacology and Experimental Therapeutics 278(3):1419-1427; Gerster et al. (1998) Analytical Biochemistry 262:177-184; Haupt et al. (1983) Journal of Chromatography 260:419-427; Shaw et al. (1995) Pharmaceutical Research 12(12)1937-1942). These methods are laborious and relatively insensitive.

Thus, there exists a need in the art for a simple, rapid, accurate, and sensitive method for quantitating nucleic acids, in particular therapeutic nucleic acids such as ribozymes, antisense oligonucleotides, or molecules that contain nucleic acids covalently attached to a protein, peptide, xenobiotic, sugar, polyethylene glycol, etc., in biological fluid samples to aid non-clinical animal safety studies, pharmacokinetic studies, etc., in biological fluid samples to aid in patient monitoring and optimization of treatment regimens in which such molecules are employed as therapeutics.

### SUMMARY OF THE INVENTION

Accordingly, in response to this need, the present inventors have developed a number of convenient methods for detecting and/or quantitating nucleic acids in biological matrices possessing the advantages of simplicity, speed, accuracy, sensitivity, and reproducibility.

Therefore, in a first aspect, the present invention provides a method for determining the concentration of a target nucleic acid in a sample of biological fluid, comprising:

(a) mixing:

(i) a sample of biological fluid suspected of containing said target nucleic acid;

(ii) a capture oligonucleotide complementary to a first region of said target nucleic acid,

wherein said capture oligonucleotide comprises a first member of a specific high affinity ligand pair; and

(iii) a detection oligonucleotide complementary to a second region of said target nucleic acid,

wherein said detection oligonucleotide comprises a detectable label moiety; and

(iv) a detergent effective in preventing or disrupting non-specific interaction between said target nucleic acid and other components of said biological fluid;

(b) if said target nucleic acid contains secondary, tertiary, or quaternary structure, heating the resulting mixture of step (a) to a temperature sufficient to disrupt said secondary, tertiary, or quaternary structure in said target nucleic acid;

(c) incubating said mixture to permit annealing of said capture and detection oligonucleotides to said target nucleic acid to form a hybridization complex between said target nucleic acid and said capture and detection oligonucleotides;

(d) binding said complex to a substrate comprising the second member of said specific high affinity ligand pair of step (a)(ii) and removing any unbound material;

(e) determining the amount of complex bound in step (d) by measuring the amount of said detectable label moiety or product thereof; and

(f) determining the concentration of said target nucleic acid in said biological fluid by comparing the amount of said detectable label moiety with a standard curve of said target nucleic acid determined in identical biological fluid or a representative surrogate matrix.

In a second aspect, the present invention provides a method for determining the concentration of each one of two or more target nucleic acids in a sample of biological fluid, comprising:

(a) mixing:

(i) a sample of biological fluid suspected of containing said two or more target nucleic acids;

(ii) a capture oligonucleotide complementary to a first region of each one of said target nucleic acids,

wherein said capture oligonucleotide comprises a first member of a specific high affinity ligand pair; and

(iii) a detection oligonucleotide complementary to a second region of each one of said target nucleic acids,

wherein each detection oligonucleotide comprises a different detectable label moiety; and

(iv) a detergent effective in preventing or disrupting non-specific interaction between each of said target nucleic acids and other components of said biological fluid;

(b) if any one of said target nucleic acids contains secondary, tertiary, or quaternary structure, heating the resulting mixture of step (a) to a temperature sufficient to disrupt said secondary, tertiary, or quaternary structure in said target nucleic acid;

(c) incubating said mixture to permit annealing of said capture and detection oligonucleotides to their respective target nucleic acid to form individual hybridization complexes between said target nucleic acids and their respective capture and detection oligonucleotides;

(d) binding each of said complexes to a substrate comprising the second member of said specific high affinity ligand pair of step (a)(ii) and removing any unbound material;

(e) determining the amount of each complex bound in step (d) by measuring the amount of said detectable label moiety or product thereof; and

(f) determining the concentration of each of said target nucleic acids in said biological fluid by comparing the amount of said detectable label moiety specific to each of said target nucleic acids with standard curves of each of said target nucleic acids determined in identical biological fluid or a representative surrogate matrix.

In a third aspect, the present invention provides a method for determining the amount of a metabolite of a target nucleic acid in a biological fluid, comprising:

(a) mixing:

(i) a sample of biological fluid suspected of containing said metabolite of said target nucleic acid;

(ii) a capture oligonucleotide complementary to a first region of said metabolite of said target nucleic acid,

wherein said capture oligonucleotide comprises a first member of a specific high affinity ligand pair; and

(iii) a detection oligonucleotide complementary to a second region of said metabolite of said target nucleic acid,

wherein said detection oligonucleotide comprises a detectable label moiety; and

(iv) a detergent effective in preventing or disrupting non-specific interaction between said metabolite of said target nucleic acid and other components of said biological fluid;

(b) if said metabolite of said target nucleic acid contains secondary, tertiary, or quaternary structure, heating the resulting mixture of step (a) to a temperature sufficient to disrupt said secondary, tertiary, or quaternary structure;

(c) incubating said mixture to permit annealing of said capture and detection oligonucleotides to said metabolite of said target nucleic acid to form a hybridization complex between said metabolite of said target nucleic acid and said capture and detection oligonucleotides;

(d) binding said complex to a substrate comprising the second member of said specific high affinity ligand pair of step (a)(ii) and removing any unbound material;

(e) determining the amount of said complex bound in step (d) by measuring the amount of said detectable label moiety or product thereof; and

(f) determining the concentration of said metabolite of said target nucleic acid in said biological fluid by comparing the amount of said detectable label moiety with a standard curve of said metabolite determined in identical biological fluid or a representative surrogate matrix.

In this method, the metabolite of said target nucleic acid can be a truncated fragment of said target nucleic acid produced by in vivo catabolism of said target nucleic acid.

In another aspect, the present invention provides a method for determining the amounts of multiple metabolites of a target nucleic acid in a biological fluid, comprising:

(a) mixing:

(i) a sample of biological fluid suspected of containing multiple metabolites of a target nucleic acid;

(ii) a capture oligonucleotide complementary to a first region of each one of said multiple metabolites of said target nucleic acid,

wherein each one of said capture oligonucleotides comprises a first member of a specific high affinity ligand pair; and

(iii) multiple detection oligonucleotides, each one of which is separately and distinctly complementary to a second region of each individual metabolite of said target nucleic acid,

wherein each one of said multiple detection oligonucleotides comprises a different detectable label moiety; and

(iv) a detergent effective in preventing or disrupting non-specific interaction between said metabolites of said target nucleic acid and other components of said biological fluid;

(b) if any of said metabolites of said target nucleic acid contains secondary, tertiary, or quaternary structure, heating the resulting mixture of step (a) to a temperature sufficient to disrupt said secondary, tertiary, or quaternary structure;

(c) incubating said mixture to permit annealing of said capture and detection oligonucleotides to said metabolites of said target nucleic acid to form hybridization complexes between said metabolites of said target nucleic acid and their respective capture and detection oligonucleotides;

(d) binding said complexes to a substrate comprising the second member of said specific high affinity ligand pair of step (a)(ii) and removing any unbound material;

(e) determining the amount of each one of said complexes bound in step (d) by measuring the amount of each one of said different detectable label moieties or product thereof; and

(f) determining the concentration of each one of said multiple metabolites of said target nucleic acid in said biological fluid by comparing the amount of each one of said different detectable label moieties specific to each one of said detection oligonucleotides with that of a standard curve of each one of said multiple metabolites determined in identical biological fluid or a representative surrogate matrix.

This method can further comprise using multiple capture oligonucleotides comprising the same specific high affinity ligand, different specific high affinity ligands, or mixtures thereof. In addition, the binding of step (d) can comprise using the same substrate or different substrates.

In yet another aspect, the present invention provides a method for detecting a metabolite of a target nucleic acid in a biological fluid, comprising:

(a) mixing:

(i) a sample of biological fluid suspected of containing said metabolite of said target nucleic acid;

(ii) a capture oligonucleotide complementary to a first region of said metabolite of said target nucleic acid,

wherein said capture oligonucleotide comprises a first member of a specific high affinity ligand pair; and

(iii) a detection oligonucleotide complementary to a second region of said target nucleic acid,

wherein said detection oligonucleotide comprises a detectable label moiety; and

(iv) a detergent effective in preventing or disrupting non-specific interaction between said metabolite of said target nucleic acid and other components of said biological fluid;

(b) if said metabolite of said target nucleic acid contains secondary, tertiary, or quaternary structure, heating the resulting mixture of step (a) to a temperature sufficient to disrupt said secondary, tertiary, or quaternary structure;

(c) incubating said mixture to permit annealing of said capture and detection oligonucleotides to said metabolite of said target nucleic acid to form a hybridization complex between said metabolite of said target nucleic acid and said capture and detection oligonucleotides;

(d) binding said complex to a substrate comprising the second member of said specific high affinity ligand pair of step (a)(ii) and removing any unbound material; and

(e) detecting the presence of said complex bound in step (d) by detecting said detectable label moiety or product thereof.

In this method, the metabolite of said target nucleic acid can be a truncated fragment of said target nucleic acid produced by in vivo catabolism of said target nucleic acid.

In yet another aspect, the present invention provides a method for detecting multiple metabolites of a target nucleic acid in a biological fluid, comprising:

(a) mixing:

(i) a sample of biological fluid suspected of containing multiple metabolites of a target nucleic acid;

(ii) a capture oligonucleotide complementary to a first region of each one of said multiple metabolites of said target nucleic acid,

wherein each one of said capture oligonucleotides comprises a first member of a specific high affinity ligand pair; and

(iii) multiple detection oligonucleotides, each one of which is separately and distinctly complementary to a second region of each individual metabolite of said target nucleic acid,

wherein each one of said multiple detection oligonucleotides comprises a different detectable label moiety; and

(iv) a detergent effective in preventing or disrupting non-specific interaction between said metabolites of said target nucleic acid and other components of said biological fluid;

(b) if any of said metabolites of said target nucleic acid contains secondary, tertiary, or quaternary structure, heating the resulting mixture of step (a) to a temperature sufficient to disrupt said secondary, tertiary, or quaternary structure;

(c) incubating said mixture to permit annealing of said capture and detection oligonucleotides to said metabolites of said target nucleic acid to form hybridization complexes between said metabolites of said target nucleic acid and their respective capture and detection oligonucleotides;

(d) binding said complexes to a substrate comprising the second member of said specific high affinity ligand pair of step (a)(ii) and removing any unbound material; and

(e) detecting the presence of each one of said complexes bound in step (d) by detecting each one of said different detectable label moieties or product thereof.

In this method, each one of said multiple capture oligonucleotides can comprise the same specific high affinity ligand, a different specific high affinity ligand, or a mixture thereof. That is, the multiple capture oligonucleotides can comprise a mixture wherein a portion of the capture oligonucleotides is each individually labelled with the same specific high affinity ligand, while the remaining portion of the capture oligonucleotides is labelled with a different specific high affinity ligand. Furthermore, in this method, the binding of step (d) can comprise using the same substrate, or different substrates.

In any one of the foregoing methods, the biological fluid can be blood, serum, saliva, urine, plasma, cerebrospinal fluid, follicular fluid, allantoic fluid, interstitial fluid, labyrinthine fluid, pericardial fluid, ventricular fluid, serous fluid, synovial fluid, tissue fluid, milk, seminal fluid, ocular fluid, amniotic fluid, placental fluid, ascitic fluid, pleural fluid, sputum, bronchial aspirate, or macerated tissue.

In any one of the foregoing methods, the length of each of said capture and detection oligonucleotides can be at least about 5 nucleotides.

In any one of the foregoing methods, the members of the specific high affinity ligand pair can be selected from the group consisting of biotin-streptavidin, an antibody-antigen, a receptor-ligand, an antibody-protein A, an antibody-protein G, a lectin-receptor, and a drug-receptor.

In any one of the foregoing methods, the detectable label moiety can be selected from the group consisting of a peptide, polypeptide, or protein; a fluorescent label; a bioluminescent label; a chemiluminescent label; a radioisotope; and a drug for which there exists a high affinity ligand. The peptide, polypeptide, or protein can detected by a method selected from the group consisting of reaction with an enzyme-linked antibody, gamma or scintillation counting, luminometry, phosphorescence, spectrophotometry, or spectrofluorometry. The enzyme-linked antibody can be selected from the group consisting of an alkaline phosphatase-conjugated antibody, a horseradish peroxidase-conjugated antibody, a β-galactosidase-conjugated antibody, and a glucose oxidase-conjugated antibody.

In any one of the foregoing methods, the detergent can be selected from the group consisting of an anionic detergent, a cationic detergent, a nonionic detergent, and a zwitterionic detergent. More particularly, the detergent can be selected from the group consisting of sodium dodecyl sulfate, deoxycholic acid (3α,12α-dihydroxy-5β-cholan-24-oic acid), N-lauroyl-sarcosine, dodecyltrimethylammonium bromide, methylbenzethonium chloride (N,N-dimethyl-N-[2-(2-[methyl-4-(1,1,3,3-tetramethylbutyl)-phenoxy]-ethoxy)ethyl]benzylammonium chloride]; triton X-100 (t-octylphenoxypolyethoxyethanol), tween-20 (polyoxyethylene-sorbitan monolaurate); and CHAPS (3-[(3-cholanimidopropyl)-dimethyl-ammonio]-1-propane-sulfonate); or N-octyl-N-N-dimethyl-3-ammonio-1-propanesulfonate.

Furthermore, in any one of the foregoing methods, the temperature sufficient to disrupt any secondary or higher structure in said target nucleic acid can be in the range of from about 60° C. to about 100° C.

Furthermore, in any one of the foregoing methods, the substrate can be selected from the group consisting of a microtiter plate, a membrane, a filter, a glass bead, a plastic bead, a metal particle, a glass slide, a plastic slide, a glass tube, a plastic tube, a latex bead, and a latex sheet.

In any one of the foregoing methods, the unbound material can be removed by washing with a buffer, centrifugation, filtration, aspiration, decantation, or absorption.

In any one of the foregoing methods, the amount of the detectable label can determined spectrophotometrically, spectrofluorometrically, by scintillation counting, gamma counting, by phosphorescence, by bioluminescence, or by chemiluminescence.

In any one of the foregoing methods, the target nucleic acid can be an oligonucleotide or a polynucleotide. More specifically, the target nucleic acid can be a therapeutically useful nucleic acid selected from the group consisting of a ribozyme; an antisense oligonucleotide; and a molecule comprising a nucleic acid covalently attached to a peptide, polypeptide, or protein; a non-peptide, polypeptide, or protein drug; a small organic molecule or drug; a sugar, for example, galactose-ribozyme; or polyethylene glycol, for example, a PEG-conjugated ribozyme. More particularly, the therapeutically useful nucleic acid can be selected from the group consisting of an anti-hepatitis C virus ribozyme, an anti-hepatitis C virus antisense oligonucleotide, an anti-angiogenesis ribozyme, an anti-angiogenesis antisense oligonucleotide, an anti-HIV ribozyme, an anti-HIV antisense oligonucleotide, an anti-influenza ribozyme, an anti-influenza antisense oligonucleotide, an anti-rhinovirus ribozyme, and an anti-rhinovirus antisense oligonucleotide. Even more particularly, the anti-hepatitis C virus ribozyme can have the sequence shown in SEQ ID NO:1.

And in yet another aspect, the present invention provides a method for determining the concentration of a ribozyme in mammalian serum, comprising:

(a) mixing:

(i) a sample of mammalian serum containing a ribozyme having the sequence shown in SEQ ID NO:1;

(ii) a capture oligonucleotide having the sequence shown in SEQ ID NO:2;

(iii) a detection oligonucleotide having the sequence shown in SEQ ID NO:3; and

(iv) sodium dodecyl sulfate at a final concentration of 1% (w/v);

(b) heating the resulting mixture of step (a) to about 75° C. for about five minutes;

(c) incubating said mixture at 37° C. in 25 mM NaCl for two hours to form a hybridization complex between said target nucleic acid and said capture and detection oligonucleotides;

(d) binding said complex to a microtiter plate well comprising streptavidin and removing any unbound material;

(e) determining the amount of complex bound in step (d) by incubating said bound complex with an anti-digoxigenin alkaline phosphatase antibody and determining the corrected optical density of the resulting solution at 405 nm after addition of p-nitrophenyl phosphate; and

(f) determining the concentration of said ribozyme in said biological fluid from a standard curve of said ribozyme determined in the same manner as in (e) in otherwise identical control mammalian serum.

In this method, the mammalian serum can selected from the group consisting of monkey serum, mouse serum, rat serum, and human serum.

Further scope of the applicability of the present invention will become apparent from the detailed description provided below. However, it should be understood that the detailed description and specific examples, while indicating preferred embodiments of the present invention, are given by way of illustration only since various changes and modifications within the spirit and scope of the invention will become apparent to those skilled in the art from this detailed description.

### BRIEF DESCRIPTION OF THE DRAWINGS

The above and other aspects, features, and advantages of the present invention will be better understood from the following detailed description taken in conjunction with the accompanying drawings, all of which are given by way of illustration only, and are not limitative of the present invention.

FIG. 1 is a schematic of the hybridization-ELISA assay design discussed in Example 1, below. In panel A, the denatured ribozyme is mixed with biotin- and digoxigenin (Dig)-labeled oligonucleotides, and binding occurs by complementary base pairing. In panel B, the ribozyme:oligonucleotide complex is transferred to a microtiter plate coated with streptavidin (SA). The biotin-labeled complex binds to the streptavidin, and non-bound material is washed away. In panel C, an alkaline phosphatase (AP)-conjugated antibody against digoxigenin is added to the microtiter plate. The alkaline phosphatase catalyzes transformation of the p-nitrophenyl phosphate (PNPP) substrate to a yellow product, which is detected using a microtiter plate-reader.

FIG. 2 shows the effect of differing salt concentrations on metabolite (24-mer) and analyte (ribozyme) response in hybridization ELISA. The hybridization ELISA assay was performed with full-length ribozyme or the 24-mer putative metabolite using hybridization buffers containing NaCl concentrations ranging from 25 mM to 300 mM. Results from 300, 100, and 50 mM are shown. Open symbols represent the 24-mer, while closed symbols represent the ribozyme molecule.

FIG. 3 shows the effect of incubation temperature on hybridization-ELISA specificity for ribozyme versus the 24-mer. The hybridization ELISA assay was performed as described in Example 1, using hybridization buffer containing 50 mM NaCl (25 mM final concentration in the hybridization mixture). All incubations were performed either at room temperature or at 37° C., as indicated.

### DETAILED DESCRIPTION OF THE INVENTION

The following detailed description of the invention is provided to aid those skilled in the in practicing the present invention. Even so, the following detailed description should not be construed to unduly limit the present invention as modifications and variations in the embodiments discussed herein can be made by those of ordinary skill in the art without departing from the spirit or scope of the present inventive discovery.

The contents of each of the references cited herein are herein incorporated by reference in their entirety.

The present invention provides novel methods for accurately determining the concentration of nucleic acid molecules present in biological matrices. Of particular interest in this regard are nucleic acids of therapeutic utility, also referred to herein as “therapeutic nucleic acids,” “therapeutic polynucleotides,” “target nucleic acids,” “nucleic acid analytes,” or “polynucleotide analytes”. Non-limiting examples of nucleic acids currently being developed for therapeutic applications, and to which the methods disclosed herein are applicable, include ribozyme molecules; antisense oligonucleotides; oligonucleotide inhibitors of viral polymerases/viral replication (Chung et al. (1994) Proc. Natl. Acad. Sci. USA 91:2372-2376; PCT International Publication No. WO 00/04141); and molecules that contain polynucleotide chains covalently attached to a peptide, polypeptide, protein, xenobiotic, sugar, polyethylene glycol, etc., that may be present in biological matrices. Preferably, such therapeutic polynucleotides are at least about 10 nucleotides in length, and can range in size from about 10 nucleotides to about 500 nucleotides, or from about 10 nucleotides to about 1000 nucleotides or more in length. Such therapeutic polynucleotides can, for example, be about 10 nucleotides in length, about 15 nucleotides in length, about 20 nucleotides in length, from about 10 to about 20 nucleotides in length (strictly categorizing them as “oligonucleotides” rather than polynucleotides), about 30 nucleotides in length, etc., or from about 10 nucleotides to about 100 nucleotides in length, from about 15 nucleotides to about 80 nucleotides in length, from about 20 nucleotides to about 50 nucleotides in length, etc. Of particular interest in the context of the present invention are therapeutic nucleic acids such as ribozymes, antisense oligonucleotides, and polymerase inhibitors useful in treating bacterial, fungal, and viral infections.

Biological fluids to which the present methods are applicable include, for example, blood, serum, saliva, urine, plasma, cerebrospinal fluid, follicular fluid, allantoic fluid, interstitial fluid, labyrinthine fluid, pericardial fluid, ventricular fluid, serous fluid, synovial fluid, tissue fluid, milk, seminal fluid, ocular fluid, amniotic fluid, placental fluid, ascitic fluid, pleural fluid, sputum, bronchial aspirate, macerated tissue, etc.

Using the hybridization-detection methods disclosed herein, one need not extract the nucleic acids of interest from the sample of biological fluid, sensitivity is greatly increased, and assay of the analyte nucleic acids can be completed within a single day, as compared to multiple days required in previous methods.

The assay methods described herein provide novel approaches to the tasks of detecting and quantifying a therapeutic nucleotide-based drug in biological samples, or metabolites thereof, and possess a number of advantages over conventional techniques.

First, the present hybridization-detection methods do not require an extraction step, which represents a major advantage in terms of speed, cost, recovery, and therefore accuracy of the assay.

Secondly, the sensitivity and specificity of the assay can be varied by varying the annealing conditions, e.g., salt concentration and temperature.

Variation in recovery of the analyte from different matrix sources was initially a problem in the studies described in Example 1. The probable explanation for this phenomenon was non-specific interaction, for example non-specific binding, of the target ribozyme molecule to other components in the serum. By employing one or more detergents, such as sodium dodecylsulfate (SDS) in the hybridization mixture, such non-specific binding, or other interaction such as adsorption, entrapment, entanglement, coagulation, micelle formation, co-precipitation, etc., could be disrupted, permitting accurate recovery of samples spiked into serum from a variety of sources. This solution to the recovery problem permits the use of the hybridization-ELISA, and other methods disclosed herein, without pre-extraction of samples. Other detergents that can be employed, alone or in combination, in the methods disclosed herein for this purpose include anionic detergents or bile salts, for example deoxycholic acid (3α,12α-dihydroxy-5β-cholan-24-oic acid) and N-lauroyl-sarcosine; cationic detergents, for example dodecyl-trimethylammonium bromide, methylbenzethonium chloride (N,N-dimethyl-N-[2-(2-[methyl-4-(1,1,3,3-tetramethyl-butyl)phenoxy]ethoxy)ethyl]-benzylammonium chloride); non-ionic detergents, for example triton X-100 (t-octylphenoxy-polyethoxyethanol) and tween-20 (polyoxyethylenesorbitan monolaurate); and zwitterionic detergents, for example CHAPS (3-[(3-cholanimidopropyl)-dimethylammonio]-1-propane-sulfonate and N-octyl-N-N-dimethyl-3-ammonio-1-propanesulfonate). Additional examples of detergents useful in the present methods can be found at page 1883 of the Sigma Catalog 2000-2001. Preferably, the detergent should not only be effective in preventing or disrupting non-specific interaction between the target nucleic acid, or a metabolite thereof, and other components of the biological fluid, but also in preventing or disrupting non-specific interaction between the capture and detection oligonucleotides and components of the biological fluid as well.

Another advantage of the present hybridization-detection assays is the use of two or more oligonucleotides to form a “sandwich” for capture and detection of the nucleic acid analyte. Using multiple, different “detection oligonucleotides” comprising different detectable label or tag moieties, one can simultaneously assay for different nucleic acid analytes in a single sample of biological fluid. In a previously published method (deSerres et al. (1996) Analytical Biochemistry 233:228-233), researchers used hybridization in a competitive scintillation proximity assay format. This approach required the use of a radiolabeled tracer for signal generation and detection, which can be a major drawback due to the problems associated with handling and disposing of radioactive material. This assay method is performed in the competitive mode, meaning that the analyte and labeled tracer compete for binding to an oligonucleotide that is immobilized on a bead. In the absence of analyte, tracer binds maximally. Signal is decreased in the presence of analyte in the sample of interest. In comparison, in the example disclosed hereinbelow, the use of separate oligonucleotide probes for capture and detection insured that small fragments of the ribozyme which may arise as products of metabolism in vivo (“metabolites” of the original target nucleic acid), are not detected, eliminating this source of interference with the accuracy of the assay. This feature is not present in the deSerres assay, in which fragments of the parent analyte would be expected to produce a significant signal in the assay.

In the methods disclosed herein, one can employ multiple, non-overlapping detection oligonucleotides, such that the capture oligonucleotide is complementary to a first region of the analyte polynucleotide, and each of two (or more) differently labeled detection oligonucleotides is complementary to second and third, etc., distinct regions, respectively, of the analyte polynucleotide. By quantifying the signal resulting from each of the two (or more) detection oligonucleotides, one can determine the amounts of full-length fragments, or metabolites thereof, such as truncated fragments of the analyte produced by metabolic degradation in vivo. Thus, the present invention provides sensitive, discriminating methods for studying the metabolism of target nucleic acids by facilitating characterization of the metabolic products thereof formed in the body over time. Such information can be useful in designing therapeutic polynucleotides with increased metabolic stability. While it is preferred that the detection oligonucleotides used in these embodiments hybridize to completely distinct, non-overlapping regions of the target nucleic acid, some degree of sequence overlap, for example from about 3 or about 5 to about 10 nucleotides, between the regions different detection oligonucleotides hybridize to in the polynucleotide analyte can be tolerated, depending upon the information being sought.

Alternately, one can multiplex, and perform analyses for multiple polynucleotide analytes simultaneously. In this case, capture and detection oligonucleotide pairs specific for various polynucleotide analytes are used together. Detection oligonucleotides can contain unique labeling moieties that permit quantitation of a polynucleotide analyte without interference from the signal of other detection oligonucleotides, and therefore other nucleic acid analytes.

A number of different molecules known in the art can be used to specifically label or tag the detection oligonucleotides used in the methods disclosed herein. Examples of such molecules include peptides, polypeptides, and proteins; enzymatic labels, in which the conjugated enzyme produces a detectable product from a substrate, such as alkaline phosphatase, horseradish peroxidase, β-galactosidase, and glucose oxidase; fluorescent labels such as fluorescine, rhodamine, Texas red, green fluorescent protein, and phycobiliproteins; chemiluminescent labels; bioluminescent labels; phosphorescent labels; radioactive nuclide labels such as ¹²⁵I, ³²P, ¹⁴C, and ³H; biotin; amine groups; a drug for which there exists a high affinity ligand, for example phenylzine and monoamine oxidase, and burimamide and the H2 receptor, etc. Labeled oligonucleotides are readily commercially available. For example, Sigma-Genosys (Woodlands, Tex.), provides such oligonucleotides. Labeling groups, for example biotin and amine groups, can be incorporated during nucleotide synthesis (L. Townsend, Ed., (1988) Chemistry of Nucleosides and Nucleotides, Plenum Press, NY. Labels can also be linked to detection oligonucleotides by post-synthetic modification (Hermanson (1996) Bioconjugate Techniques, Academic Press, New York; Ruth (1984) DNA 3:12). The label can be conjugated to the oligonucleotide covalently or non-covalently.

Detection of the bound complex is achieved either directly or indirectly. Direct detection refers to the case where the detection oligonucleotide is labeled with a group that contains or creates a measurable signal, such as a radioactive nuclide, fluorescent group, or enzyme which produces a measurable product. Indirect detection is achieved when the detection oligonucleotide is labeled with a group that itself may be bound by specific high affinity ligand labeled in such a way to permit detection (a radioactive nuclide, a fluorescent group, an enzyme which produces a measurable product).

As used herein, the phrase “product of said detectable label moiety” refers to the situation wherein the label moiety is not itself directly detected, but rather catalyzes or otherwise facilitates a reaction that generates a product which is detected. For example, in the example provided below, the detectable label moiety is digoxigenin, which is indirectly detected by binding of an anti-digoxigenin antibody conjugated to alkaline phosphatase, which catalyzes the conversion of p-nitrophenyl phosphate to a colored product, which is detected calorimetrically.

The capture oligonucleotides can be conjugated to any molecule for which there exists a specific high affinity ligand (Hermanson et al. (1992) Immobilized Affinity Ligand Techniques, Academic Press, NY). Non-limiting examples of high affinity ligand pairs useful in the present methods include biotin/avidin (Gitlin et al. (1987) Biochemical Journal 242:923-926); antibody/antigen pairs (Harlow et al. (1988) Antibodies: A Laboratory Manual, Cold Spring Harbor Laboratory Press, NY, pp. 23-35); antibody/protein A or protein G pairs (Harlow et al. (1988) Antibodies: A Laboratory Manual, Cold Spring Harbor Laboratory Press, NY, pp 616-623); ligand/receptor pairs, for example insulin and the insulin receptor (Gammeltoft et al. (1985) Biochimi 67:1147-53); and lectin/receptor pairs (Kocourek et al. (1981) Nature 290:188). To capture the oligonucleotide/polynucleotide analyte complex, the first member of the high affinity ligand pair is affixed or attached, covalently or non-covalently, to the binding substrate, for example by coating, while the second member of the ligand pair is conjugated covalently or non-covalently to the oligonucleotide used for capture.

Depending on the need for heating to denature any secondary or higher structure in the polynucleotide analyte of interest and the stabilty of the moieties used for capture and detection of the polynucleotide/oligonucleotide complex, the assay can be performed by adding all reagents simultaneously. Where labile capture and detection moieties are used, the capture and detection oligonucleotides can be added after denaturation of the polynucleotide analyte. After binding/hybridization, non-bound components can be removed by, for example, washing with a buffer, centrifugation, filtration, aspiration, decantation, or absorption, and results measured after addition of any substrates required for generation of a detectable signal. Alternatively, the assay can be performed in a homogeneous format, in which removal of non-bound components is unnecessary. One example of this latter format employs scintillation proximity beads, where the signal is only generated upon binding of a radiolabeled detection oligonucleotide to the captured nucleic acid. The capture oligonucleotide can be directly bound to the scintillation proximity bead, or indirectly bound as described above (for example, by biotin/streptavidin interaction).

An additional advantage of the present methods includes the ability to optimize the conditions under which the detection and capture oligonucleotides hybridize to the polynucleotide analyte or metabolite thereof, e.g., temperature and salt concentration, to increase hybridization stringency, and therefore assay specificity and sensitivity. Such optimization, for example high stringency hybridization conditions, eliminates significant cross-reactivity with related polynucleotide analytes, and/or metabolites of the specific target nucleic acid being studied. This is illustrated in the example provided below, where it was possible to discriminate between the target ribozyme and a 24-mer degradation product thereof.

Those of ordinary skill in the art are familiar with methods for varying the stringency of nucleic acid hybridization reactions. Note, for example, Sambrook et al. (1989) Molecular Cloning, A Laboratory Manual, Second Edition, Cold Spring Harbor Laboratory Press, NY, chapter 9, especially pages 9.47-9.51 (long polynucleotides), and chapter 11, especially pages 11.7-11.8 and 11.45-11.57 (shorter oligonucleotides) and Ausubel et al. (1987 and supplements) Current Protocols in Molecular Biology, John Wiley & Sons, Inc., NY, chapter 2, especially section 2.9, and chapter 6, especially sections 6.3-6.4, and the references cited therein. Stringency is a term which describes the conditions (temperature, salt concentration, etc.) under which base-pairing occurs. Higher temperature and lower salt result in increased stringency. In the assays described herein, hybridization stringency can be adjusted to alter the specificity of the assay. A high stringency condition was used in the example described below in order to decrease signal from a truncated metabolic product of the full-length ribozyme. Lower stringency (lower hybridization temperature and higher salt) can be used to quantify “total” drug (full-length ribozyme plus metabolic fragments). Alternately, lower stringency can be used if metabolic fragments are not produced. Hybridization or annealing of the capture and detection oligonucleotides to the target nucleic acid can performed in the methods of the present invention in a temperature range of from about 15° C. to about 65° C., preferably from about 22° C. to about 50° C., more preferably from about 30° C. to about 45° C., and most preferably from about 35° C. to about 40° C. Temperatures can be about 35° C., about 40° C., about 45° C., about 50° C., about 55° C., about 60° C., or about 65° C. Using the equations given in Sambrook et al. and Ausubel et al., the optimal temperature for hybridization for the particular oligonucleotides/target nucleic acid in question can be determined. The salt concentration for hybridization or annealing can be in the range of from about 25 mm to about 1 M, more preferably from about 25 mM to about 500 mM more preferably from about 25 mM to about 400 mM, more preferably from about 25 mM to about 300 mM, more preferably from about 50 mM to about 250 mM, more preferably from about 100 mm to about 200 mM. Salt concentrations can be about 25 mM, about 50 mM, about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, or about 1 M. Again, using the equations given in Sambrook et al. and Ausubel et al., one can determine the optimal salt concentration for the particular oligonucleotides/target nucleic acid in question.

Typically, time periods for nucleic acid hybridization can vary from about 15 minutes to 48 hours, preferably from about 15 minutes to about 24 hours, more preferably from about 15 minutes to about 12 hours, more preferably from about 15 minutes to about 8 hours, more preferably from about 15 minutes to about 6 hours, more preferably from about 30 minutes to about 4 hours, more preferably from about 1 hour to about 3 hours, more preferably from about 1 hour to about 2 hours. From about twelve to about 16 hours, from about 15 minutes to about 2 hours, from about 15 minutes to about 1 hour, or from about 15 minutes to about 30 minutes can also be used. As in the case of temperature and salt concentration, the time required for optimal hybridization between any particular combination of capture/detection oligonucleotides:target nucleic acid can be determined empirically by routine experimentation.

If a control sample of a biological fluid under investigation is not readily available, a representative surrogate matrix can be used in constructing standard curves for the assays described herein. In some cases, the composition of various biological fluids, e.g., protein nature and quantity, salt concentration, lipid nature and quantity, etc., is known. In these instances, one of ordinary skill in the art can mix individually commercially available components to prepare a solution that closely resembles the biological fluid in question. In the case where the composition of the biological fluid is less well characterized, one can, for example, empirically identify a surrogate matrix that performs similarly to the biological fluid in question in the methods disclosed herein by carrying out appropriate comparative experiments between the two.

Commercial sources of biological fluids include Bioreclamation Inc. (East Meadow, N.Y.) and Pel-Freez Biologicals (Rogers, Ariz.).

A number of molecular biological techniques are designed to take advantage of the specific base-pairing of RNA or DNA molecules. These techniques include cloning, in situ hybridization, PCR (polymerase chain reaction), Northern blotting, and Southern blotting. These methods possess a variety of limitations in terms of sensitivity, and are only semi-quantitative. Recently, commercial kits such as the R&D Systems Quantikine mRNA or the Chemicon OligoDetect assays have been developed to quantify mRNA (message RNA) or PCR products using capture/detection techniques. These approaches, however, are not directly applicable to measurement of a therapeutic drug in biological matrices. The Chemicon assay uses one hybridization probe, which limits specificity. In this assay, truncated fragments of the mRNA will cross-react in the assay. As a result, the RNA quantitated will include the amount of functional full-length RNA, plus fragments, which may not be functional. The Chemicon assay also requires incorporation of biotin in a PCR product for detection. For small (less than 50 nucleotides) molecules, PCR is not effective due to the constraints in designing two specific probes that will not amplify other RNA or DNA species. Both the Chemicon and R&D assays have the drawback of requiring significant sample pre-treatment prior to analysis, which limits sample throughput.

The R&D Quantikine mRNA assay is designed to quantify a specific mRNA that has been extracted from cells or tissues, and uses two differently labeled oligonucleotide probes for mRNA capture and detection. Because mRNA species are large (generally thousands of bases in length), specificity can be driven by using long oligonucleotide probes. In contrast, in the hybridization-ELISA assay disclosed in Example 1, two probes hybridizing over as few as 16 nucleotides will bind specifically to a target nucleic acid. Specificity can be further increased by altering the hybridization temperature and the salt concentration in the hybridization buffer, as discussed above.

The novel methods disclosed herein can be used to measure concentrations of therapeutic nucleic acids (RNA or DNA) in biological matrices. The “sandwich” format of the present hybridization-ELISA assay permits the use of non-isotopic detection for signal generation. With design of appropriate capture/detect oligonucleotides, this assay format is broadly applicable to analysis of therapeutic nucleic acids without significant sample pre-treatment.

It should be noted that while the present invention provides a number of different methods for quantitating the amount of a target nucleic acid, or metabolite(s) thereof, present in a sample of biological fluid, qualitative methods that only provide information on the presence of such nucleic acids or metabolite(s) in biological fluids are also provided.

In addition, the present invention is useful in pharmacokinetic studies as it provides methods for monitoring quantitative and qualitative aspects of drug metabolism in vivo. By performing successive assays over time, for example once every about 72 hours, once every about 48 hours, once every about 24 hours, once every about 12 hours, once every about 8 hours, once every about 6 hours, once every about 4 hours, or once every about 2 hours, one can, using the methods provided herein, quantitate the amount of therapeutic nucleic acid remaining in the body, i.e., the time course of degradation. In addition, in assays employing multiple, non-overlapping detection oligonucleotides complementary to different, distinct regions of the therapeutic nucleic acid in the present assays over similar time periods, one can monitor the nature (structure) of fragments of the original therapeutic nucleic acid appearing or remaining over time, i.e., the pathway of degradation. Such information relating to the catabolism of the therapeutic nucleic acid can provide guidance as to how to the nucleic acid can be structurally modified in order to improve its stability, and therefore therapeutic effectiveness, in vivo.

In a specific embodiment, the present assay method comprises the following steps. Serum or other biological fluid containing the therapeutic nucleic acid analyte, for example a hammerhead ribozyme, is mixed with biotin- and digoxigenin-labeled DNA oligonucleotides complementary to the two halves of the ribozyme molecule. Labeled RNA oligonucleotides can also be used in the methods disclosed herein. The serum/oligonucleotide mixture is heated at about 75° C. for approximately 5 minutes to disrupt the secondary structure in the ribozyme, and then incubated at 37° C. to permit annealing of the labeled oligonucleotides to the ribozyme. The ribozyme hairpin loop could be denatured at 75° C. to create access for the capture/detection oligonucleotides described herein, surprisingly without protein aggregation and precipitation at this temperature. The ribozyme-oligonucleotide complex is captured by binding to a streptavidin-coated microtiter plate, and detected with an anti-digoxigenin alkaline phosphatase-conjugated antibody. The substrate p-nitrophenyl phosphate (PNPP) is then added to the complex, and is converted by alkaline phosphatase to a yellow product which is quantified calorimetrically at 405/485 nm using a microtiter plate absorbance reader. Assay data are analyzed by computer using a fixed weighted 4/5-parameter logistic algorithm. For this assay, the concentration of ribozyme in test samples was determined from a standard curve of ribozyme prepared in serum ranging in concentration from 0.37 to 270 ng/mL. For the human, mouse, and monkey serum assays, the validated lower and upper limits of quantitation (LLOQ and ULOQ) were 5.0 and 120 ng/mL, respectively.

Capture and detection oligonucleotides must be long enough to form specific and stable binding interactions, i.e., hybridize by base pairing, with the complementary regions of the polynucleotide analyte. The hybridizing portion of capture and detection oligonucleotides should preferably be at least about 5 nucleotide residues in length, more preferably at least about 7 nucleotides in length, more preferably at least about 10 nucleotides in length, more preferably at least about 15 nucleotides in length, more preferably from about 15 to about 20 nucleotides in length, and even more preferably at least about 20 nucleotides in length. While the capture and detection oligonucleotides are ideally fully (100%) complementary over their entire length to different regions of the target nucleic acid, this is not absolutely required. It is only necessary that the hybridizing region of the capture and detection oligonucleotides meet the minimum complementary nucleotide requirements set forth above. Thus, the capture and detection oligonucleotides can comprise regions of non-complementarity to the regions of the polynucleotide analyte to which they hybridize.

If the analyte nucleic acid contains secondary, tertiary, or quaternary structure that must denatured in order to permit efficient oligonucleotide binding, the following ranges of temperatures can be used: from about 60° C. to about 100° C., preferably from about 65° C. to about 95° C., more preferably from about 70° C. to about 90° C., more preferably from about 75° C. to about 85° C., and most preferably from about 75° C. to about 80° C.

Incubation temperatures employed during nucleic acid hybridization, antibody binding, and reaction product formation will vary depending on the melting temperature of the oligonucleotide/nucleotide analyte duplex, stability of the antibody, optimal temperature for reaction, etc. The stability of the labeled (or labelling) groups may be affected by increased temperature, and this should therefore be taken into account. For example, nucleic acid hybridizations and other incubations can performed in a temperature range of from about 15° C. to about 65° C., preferably from about 22° C. to about 50° C., more preferably from about 30° C. to about 45° C., most preferably from about 35° C. to about 40° C. Oligonucleotide/target nucleic acid hybridization can be optimized as discussed above.

The following non-limiting example illustrates various aspects of the present invention. However, the invention should not be considered to be limited thereby.

### EXAMPLE 1

### Quantitation of a Hammerhead Ribozyme in Mammalian Serum

An experiment in which a hammerhead ribozyme was quantitated in mammalian (monkey) serum was carried out in order to demonstrate the accuracy, reproducibility, and specificity of the hybridization-detection principles and methods disclosed herein.

Materials

The hammerhead ribozyme molecule had the following nucleotide sequence (SEQ ID NO:1):

5′-ccaagacUGAuGaggcguuagccGaaAggaccB-3′

This molecule normally exists in a tightly bound stem-loop configuration, as shown below:

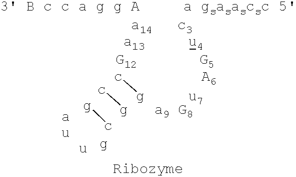

Lower case letters indicate 2′-O-methyl nucleotides. “S” indicates phosphorothioate linkages. Upper case letters indicate native ribonucleotides. “B” indicates inverted abasic residues (Macejak et al. (2000) Hepatology 31:769-776).

DNA oligonucleotides complementary to both halves of the ribozyme molecule used to capture (SEQ ID NO:2) and detect (SEQ ID NO:3) the ribozyme were custom synthesized by Sigma-Genosys (Woodlands, Tex.), and had the following sequences:

SEQ ID NO:2 (Capture Oligonucleotide)

3′-biotin-ggttctgactactccg-5′

SEQ ID NO:3 (Detection Oligonucleotide)

3′-caatcggctttcctggaa-Digoxigenin-5′

The hybridization complex formed between the ribozyme and the capture and detection oligonucleotides had the following structure:

```
Ribozyme: 5′ ccaagacUGAuGaggcguuagccGaaAggaccB 3′
        3′biotin-ggttctgactactccgcaatcggctttcctggaa-
        Dig 5′
```

The upper line shows the sequence of the ribozyme (SEQ ID NO:1). The lower line shows the sequence of the biotinylated capture oligonucleotide (SEQ ID NO:2) and the digoxigenin-conjugated detection oligonucleotide (SEQ ID NO:3). The capture oligonucleotide is shown underlined; the detection oligonucleotde is in italics. Each DNA oligonucleotide was complementary to half of the sequence of the ribozyme molecule.

A 3′ biotin moiety was incorporated in the capture oligonucleotide (SEQ ID NO:2: 5′-gcc tca tca gtc ttg g-Biotin-3′) during synthesis. A 5′ digoxigenin moiety was added to the detection oligonucleotide (SEQ ID NO:3: 5′-Digoxigenin-aag gtc ctt tcg gct aac-3′) post-synthetically by the vendor. Additionally, two non-hybridizing adenine residues were added at the 5′ end of the sequence of the detection oligonucleotide to improve antibody binding by reducing steric hindrance.

Streptavidin-coated microtiter plates (transparent, 96-well) and anti-digoxigenin alkaline phosphatase conjugate (anti-Dig-AP FAB fragment) were purchased from Roche Molecular Biochemicals, Inc. (Indianapolis, Ind.). Cluster tube 8-well strips were from Costar (Cambridge, Mass.).

ImmunoPure PNPP substrate and 5× diethanolamine substrate buffer were purchased from Pierce (Rockford, Ill.). Tris base, magnesium chloride, sodium dodecyl sulfate, EDTA (ethylenediamine tetra-acetic acid, dipotassium salt), and sodium chloride were RNAse-free molecular biology grade from Sigma Chemical Co. (St. Louis, Mo.). DEPC (diethyl pyrocarbonate) and sodium hydroxide were reagent grade from Sigma Chemical Co. Water used in preparation of stock solutions or buffers (except ELISA wash buffer) was pre-treated with DEPC to remove RNAse contamination. Alternately, RNAse-free water was purchased Sigma Chemical Co. (St. Louis, Mo.) and used instead of DEPC-treated water.

Methods

For preparation of standards and control samples, the ribozyme was suspended in DEPC-treated water at a concentration of 1 mg/mL. For calibration standards, this stock was then serially diluted into serum (human, mouse, or monkey) to concentrations of 270, 90, 30, 10, 3.3, 1.1, and 0.37 ng/mL. Sera were purchased from SeraCare (Oceanside, Calif.), or were prepared from whole blood. Serum generation from whole blood was performed as follows. Blood was collected and allowed to clot at room temperature for approximately 30 minutes. After clotting, blood samples were centrifuged at 3000× g for 10 minutes. Serum (the fluid supernatant) was removed from the clots for use in assays. Pools of serum were generated by mixing the sera from multiple individual animals or humans. Control samples were prepared by spiking analyte initially dissolved in water into serum at the following defined concentrations: 120, 80, 40, and 5 ng/mL.

Each of the DNA oligonucleotides was resuspended at a concentration of 100 μg/mL in DEPC-treated water. Oligonucleotides were then further diluted to a concentration of 1 μg/mL in hybridization buffer (20 mM Tris-HCl, 50 mM NaCl, 0.5 mM MgCl₂, pH 7.4). Serum dilution buffer (120 μL of 20 mM Tris, 2.5% SDS (w/v), and 0.5 mM MgCl₂) was added to each well of the cluster tubes, followed by addition of 30 μL of standard, control, or sample. It should be noted that the range of quantitation of the assay can be altered by changing the volume of the serum dilution buffer and serum added in the assay. A volume of 75 μL of each of the 1 μg/mL oligonucleotides was added to the diluted serum, after which the tubes were covered and heated at 75° C. for 5 minutes in a water bath. After heating, the tubes were removed from the water bath and allowed to cool for approximately 5 minutes at room temperature. A 100 μL volume of the hybridization mix was then transferred to duplicate wells of the 96-well streptavidin microtiter plate. Plates were sealed with an adhesive plate cover and placed on a rotating shaker (˜450 rpm) at 37° C. for 2 hours. Non-bound material was removed with five 5-second washes using an EL404 plate washer (20 mM Tris Base, 150 mM NaCl, 0.1% Tween-20, pH 10.0). The anti-Dig-AP antibody was diluted to 250 mU/mL in hybridization buffer, and 100 μL were added to each well. The plate was then incubated for 1.5 hours at 37° C. with shaking at ˜450 rpm. Following the antibody binding step, the plate was again washed as described above. PNPP substrate was prepared with the ethanolamine buffer as recommended by the manufacturer, and 100 μL were added to each well. Development of the 270 ng/mL standard was monitored at 405 nm. When this standard reached an optical density of approximately 2.5, development was stopped by the addition of 25 μL of 20% EDTA to each well. Plates were read at 405 and 485 nm, and the 485 reference readings were subtracted from the reading at 405. Data were analyzed using a 5 parameter logistic algorithm (StatLia, Brendan Scientific). Other mathematical algorithms that can be employed to describe the concentration versus response data include linear, 3- or 4-parmeter logistic, and cubic spline curves (Rodgers (1981) Quality Control and Data Analysis in Binder-ligand Assay, Anaheim Calif.; Scientific Newsletter, Vol. I and II.; Rodbard (1978) in Clinical Immunochemistry, American Association for Clinical Chemistry, Washington D.C., pp 447-494).

Results

Assay Design

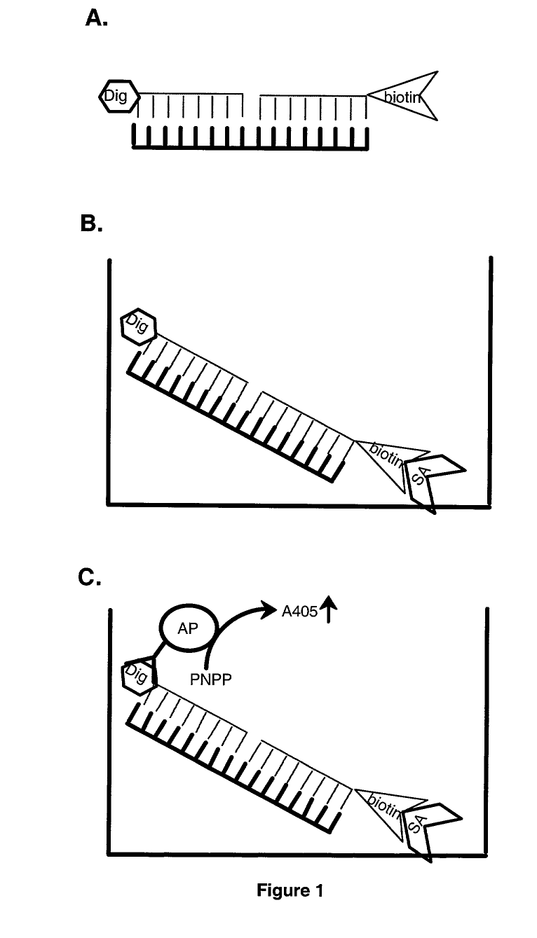

A schematic of the general assay design is shown in FIG. 1. Serum or other sample fluid is added to a diluent buffer in, for example, microtiter cluster tubes. Subsequently, the hybridization buffer containing the oligonucleotides is added, and the entire mixture is heated to denature the secondary structure in the ribozyme molecule and permit binding of the capture and detection oligonucleotides (FIG. 1a). The assay mixture is then transferred to streptavidin-coated microtiter plates for hybridization and binding of the oligonucleotide-ribozyme complex to the streptavidin (FIG. 1b). After washing to remove non-bound materials, the anti-Digoxeginin-alkaline phosphatase (AP) antibody is used to detect the bound complex (FIG. 1c).

The biotin and digoxigenin-labeled oligonucleotides used in the present example were designed to have comparable melting temperatures in order to bind with equal strength with the ribozyme molecule. Reduction in the size of these oligonucleotides resulted in lack of reproducibility in binding. Capture and detection oligonucleotides should be long enough to form specific and stable binding interactions with the polynucleotide analyte. The hybridizing portion of capture and detection oligonucleotides should preferably be at least about 5 contiguous nucleotide residues in length, more preferably at least about 7 contiguous nucleotides in length, more preferably at least about 10 contiguous nucleotides in length, more preferably at least about 15 contiguous nucleotides in length, more preferably between about 15 to about 20 contiguous nucleotides in length, and even more preferably at least about 20 contiguous nucleotides in length. These oligonucleotides can be fully, i.e., 100%, complementary along their own length to the hybridizing region along the polynucleotide analyte, or they can be partially, i.e., less than 100% complementary along their own length thereto. These oligonucleotides can, as for example in the case of SEQ ID NO:3, be longer than the complementary region of the polynucleotide target, or comprise regions that do not hybridize to a region within the analyte. Perfect and complete complementarity, while being desirable, is not required as long as the foregoing minimum length requirements are satisfied. If, for example, the target polynucleotide is 100 nucleotides in length, oligonucleotides of any of the lengths indicated above in this paragraph would probably be as effective as one another. While there is probably a minimum effective length, e.g., at least about 5 contiguous nucleotides, there is no maximum length except within the bounds of practicality, depending upon the length of the target polynucleotide.

In the present experiment, shaking in all incubation steps minimized the coefficient of variation of replicates across the plate. Other operating parameters of the assay that can be optimized to improve assay precision, accuracy, and specificity include the incubation temperature during hybridization, the salt concentration in the hybridization buffer, the time for hybridization, and the detergent to improve recovery, as described below.

Denaturation Temperature

Because the hammerhead ribozyme employed in this example normally exists in a tightly bound stem-loop configuration as shown above, it was necessary to denature this self-binding to permit efficient hybridization of the capture and detection oligonucleotides. If the analyte nucleic acid contains secondary (or higher) structure that must be denatured in order to permit oligonucleotide binding, the following temperature ranges can be used: from about 60° C. to about 100° C., preferably from about 65° C. to about 95° C., more preferably from about 70° C. to about 90° C., more preferably from about 75° C. to about 85° C., and most preferably from about 75° C. to about 80° C.

In initial attempts in the present experiment, denaturation of the ribozyme molecule in serum at 95° C. for 5 minutes resulted in aggregation and precipitation of serum proteins. The resulting precipitate could not be effectively removed by either centrifugation or filtration. In subsequent attempts, it was found that denaturation at 75° C. for 5 minutes was sufficient to unfold the secondary structure of the ribozyme without formation of protein precipitates. This denaturation condition was therefore adopted for subsequent work herein.

Effect of Salt Concentration on Metabolite Cross-Reactivity

The analyte ribozyme employed in this example was synthesized incorporating RNAse resistant nucleotides in most positions. However, not all the component ribonucleotides were modified. Based on the absence of modifications in some positions, it was predicted that one of the major (and initial) metabolites would be a 24-mer (SEQ ID NO:4: 5′-gaugaggcguuagccgaaaggaccB-3′) corresponding to the region of SEQ ID NO:1 shown in bold, below:

5′-ccaagacUGAuGaggcguuagccGaaAggaccB-3′

Optimization efforts were focused on minimizing cross-reactivity with this fragment, while still retaining good assay response with the parent ribozyme molecule.

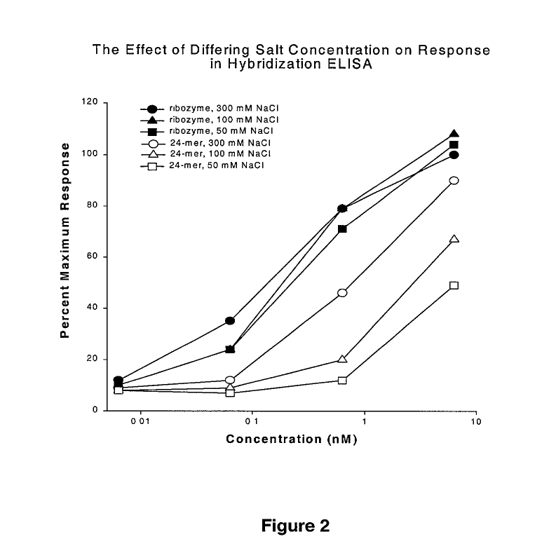

FIG. 2 shows the effect of salt concentration on response curves with full-length ribozyme and the 24-mer metabolite. Sodium chloride concentrations ranging from 300 to 25 mM were tested in the assay, and results from 300 to 50 mM are shown in the figure to illustrate the effect of salt concentration on the specificity of the assay. As the hybridization buffer was mixed 1:1 with sample in sample dilution buffer, the final salt concentration was half of that in the hybridization buffer. Results with 25 mM NaCl hybridization buffer were comparable to those with 50 mM NaCl buffer, and are not shown. For the full-length ribozyme, assay response decreased only slightly as salt concentration decreased. However, for the 24-mer metabolite, assay response was significantly diminished by reducing NaCl from 300 to 50 mM.

These results demonstrate that the ribozyme/24-mer metabolite response ratio could be maximized by reducing the sodium chloride concentration in the hybridization buffer to 50 mM.

Effect of Hybridization/Binding Temperature on Metabolite Cross-Reactivity

An additional variable that could affect the oligonucleotide specificity was the hybridization/binding temperature. Initially, binding of the oligonucleotides to the ribozyme and binding of the anti-digoxigenin-AP conjugate were performed at room temperature. Because the melting temperature (Tm) is generally higher when there are more base-pairs formed, it is theoretically possible to reduce cross-reactivity of truncated fragments in this assay by increasing the temperature at which binding occurs. Therefore, an experiment was performed examining the effect of conducting binding incubations at room temperature or at 37° C., in which the response from full-length ribozyme and the 24-mer fragment was compared. Both the initial 2 hour incubation on the streptavadin plate and the subsequent 1.5 hour incubation with the anti-digoxigenin AP conjugate were performed at the same temperature (room temperature or 37° C.).

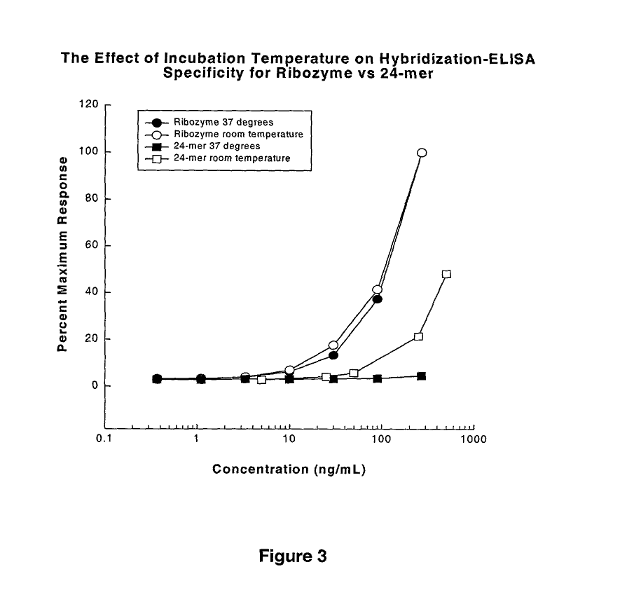

FIG. 3 shows response curves generated at room temperature and at 37° C. for the ribozyme and 24-nucleotide fragment of the ribozyme (24-mer). While there was little alteration in the response curves generated at either temperature for the full-length ribozyme molecule, response for the 24-mer was significantly decreased at 37° C. relative to room temperature. This modification of incubation temperature, in combination with determination of an appropriate salt concentration for the hybridization buffer, resulted in less than 1% cross-reactivity of the 24-mer at concentrations in the validated range of the assay.

As noted above, Sambrook et al. provides guidance as to how hybridization parameters such as salt concentration and temperature can be varied to optimize nucleic acid hybridization depending upon oligonucleotide or polynucleotide length and base composition.

Effect of Detergent on Recovery

In order to verify that the assay performed accurately in serum derived from different pooled lots or individual animals, serum samples from a variety of sources were spiked with ribozyme at 80 ng/mL. These control samples were assayed versus a standard curve prepared in serum pool 1.

As shown in Table 1, serum from some sources (individual monkeys or pooled lots) demonstrated a lack of recovery within ±20% of theoretical. In the case of one pooled lot of monkey serum, recovery was less than 10%. The addition of sodium dodecyl sulfate (SDS) to a final concentration of 1% (w/v) in the assay mixture was sufficient to improve recovery to within ±20% of the theoretical value. Results for monkey samples with and without the presence of added SDS are shown in Table 1. A similar improvement in recovery was demonstrated in mouse serum samples after the addition of SDS to the assay buffer (data not shown).

TABLE 1
Recovery of ribozyme in monkey serum measured by
hybridization assay with and without 1% SDS

| Serum | % Accuracy^{c} | % Accuracy^{c} |
|---|---|---|
| source | (−) SDS | (+) SDS |
| M-1^{a} | 33 | 115 |
| M-2 | 25 | 116 |
| M-3 | 43 | 120 |
| M-4 | 34 | 118 |
| M-5 | 26 | 105 |
| M-6 | 51 | 112 |
| M-7 | 59 | 115 |
| M-8 | 53 | 107 |
| Pool 1^{b} | 84 | 106 |
| Pool 2 | 26 | 97 |
| Pool 3 | 9 | 103 |
| Pool 4 | 36 | 103 |

^{a}Analyte was added to serum from individual monkeys, designated M1-M8.
^{b}Analyte was added to pools of serum, designated Pool 1-Pool 4. Pools of serum were created by mixing serum from several different animals.
^{c}Percent accuracy was calculated as the measured concentration divided by the theoretical (added) concentration × 100.

Validation Results

The assay described above was validated for use in mouse, human, and monkey serum. Only validation results in monkey serum are presented here, and are representative of the data obtained for all sera tested.

Table 2 shows the back-fit calculations for standard curve data used in validation runs, as well as the cumulative precision and accuracy of the back-fit calculations. Table 3 shows the overall precision and accuracy for control samples in the assay validation. As these data demonstrate, within the 5-120 ng/mL range, the overall percent coefficient of variation (%CV) and percent relative error (%RE, % accuracy-100) were less than or equal to 7.3 and 2.9%, respectively.

These data demonstrate that across the validated concentration range, results for the present hybridization-ELISA assay are accurate, with results within ±20 percent of theoretical. The assay is also precise, with a percent coefficient of variation within and between assays less than or equal to +15%. Finally, results provided by the hybridization-ELISA method are sensitive and reproducible.

TABLE 2
Back-fit Calculations for Standards in Hybridization-ELISA Assay

| Standard | | | | | | | % |
|---|---|---|---|---|---|---|---|
| Concen- | Backfit (Calculated) Concentration | | | | | % | Accu- |
| tration | Assay 1 | Assay 2 | Assay 3 | Assay 4 | Mean | CV^{a} | racy^{b} |
| 270 | 277 | 275.5 | 282.9 | 279 | 278.6 | 1.2 | 103.2 |
| ng/mL | | | | | | | |
| 90 ng/mL | 86.8 | 87.4 | 86.6 | 85.7 | 86.6 | 0.8 | 96.3 |
| 30 ng/mL | 30.8 | 31 | 29.6 | 31.3 | 30.7 | 2.4 | 92.1 |
| 10 ng/mL | 9.9 | 9.9 | 10.8 | 9.8 | 10.1 | 4.6 | 101.0 |
| 3.33 | 3.4 | 3.3 | 3.3 | 3.4 | 3.35 | 1.7 | 100.6 |
| ng/mL | | | | | | | |
| 1.11 | 1 | 1.1 | 1.1 | 1.1 | 1.1 | 4.7 | 96.8 |
| ng/mL | | | | | | | |
| 0.37 | 0.4 | 0.4 | 0.4 | 0.4 | 0.4 | 0.0 | 108.1 |
| ng/mL | | | | | | | |

^{a}% CV is the percent coefficient of variation, which is (standard deviation/mean response) × 100.
^{b}Percent accuracy was calculated as the measured concenttration divided by the theoretical (added) concentration × 100.

TABLE 3
Hybridization-ELISA Validation in Monkey Serum

| | Control (ng/mL) | | |
|---|---|---|---|
| Assay # | 5 | 40 | 120 |
| 1 | 4.5 | 37.5 | 124.1 |
| | 5.1 | 41.5 | 122.2 |
| Mean | 4.8 | 39.5 | 123.2 |
| % Accuracy^{a} | 96.1 | 98.8 | 102.6 |
| 2 | 5.2 | 39.4 | 114.1 |
| | 5.2 | 43.7 | 115.4 |
| Mean | 5.2 | 41.6 | 114.8 |
| % Accuracy | 104.8 | 103.9 | 95.6 |
| 3 | 5.0 | 41.0 | 147.0 |
| | 5.0 | 39.5 | 128.3 |
| Mean | 5.0 | 40.3 | 137.6 |
| % Accuracy | 99.4 | 100.7 | 114.7 |
| 4 | 4.9 | 38.4 | 113.5 |
| | 5.8 | 45.3 | 122.5 |
| | 5.1 | 41.5 | 122.1 |
| | 5.4 | 42.2 | 119.2 |
| | 5.1 | 40.5 | 120.5 |
| | 5.1 | 43.2 | 128.3 |
| Mean | 5.1 | 41.9 | 124.4 |
| St Dev | 0.0 | 1.9 | 5.5 |
| % CV^{b} | 0.0 | 4.5 | 4.4 |
| % Accuracy | 102.2 | 104.6 | 103.6 |
| | Inter-assay summary | | |
| Mean | 5.1 | 41.1 | 123.1 |
| St Dev | 0.3 | 2.3 | 9.0 |
| % CV | 6.1 | 5.5 | 7.3 |
| % Accuracy | 102.6 | 102.9 | 102.6 |

^{a}Percent accuracy was calculated as the measured concentration divided by the theoretical (added) concentration × 100.
^{b}% CV is the percent coefficient of variation, which is (standard devation/mean response) × 100.

The invention being thus described, it is obvious that the same can be varied in many ways. Such variations are not to be regarded as a departure from the spirit and scope of the present invention, and all such modifications as would be obvious to one skilled in the art are intended to be included within the scope of the following claims.

What is claimed is:

1. A method for determining the concentration of a target nucleic acid in a sample of biological fluid, comprising:
(a) mixing:
(i) a sample of biological fluid suspected of containing said target nucleic acid;
(ii) a capture oligonucleotide complementary to a first region of said target nucleic acid,
wherein said capture oligonucleotide comprises a first member of a specific high affinity ligand pair; and
(iii) a detection oligonucleotide complementary to a second region of said target nucleic acid,
wherein said detection oligonucleotide comprises a detectable label moiety; and
(iv) a detergent effective in preventing or disrupting non-specific interaction between said target nucleic acid and other components of said biological fluid;
(b) if said target nucleic acid contains secondary, tertiary, or quaternary structure, heating the resulting mixture of step (a) to a temperature sufficient to disrupt said secondary, tertiary, or quaternary structure in said target nucleic acid;
(c) incubating said mixture to permit annealing of said capture and detection oligonucleotides to said target nucleic acid to form a hybridization complex between said target nucleic acid and said capture and detection oligonucleotides;
(d) binding said complex to a substrate comprising the second member of said specific high affinity ligand pair of step (a)(ii) and removing any unbound material;
(e) determining the amount of complex bound in step (d) by measuring the amount of said detectable label moiety or product thereof; and
(f) determining the concentration of said target nucleic acid in said biological fluid by comparing the amount of said detectable label moiety with a standard curve of said target nucleic acid determined in identical biological fluid or a representative surrogate matrix.

2. A method for determining the concentration of each one of two or more target nucleic acids in a sample of biological fluid, comprising:
(a) mixing:
(i) a sample of biological fluid suspected of containing said two or more target nucleic acids;
(ii) a capture oligonucleotide complementary to a first region of each one of said target nucleic acids,
wherein said capture oligonucleotide comprises a first member of a specific high affinity ligand pair; and
(iii) a detection oligonucleotide complementary to a second region of each one of said target nucleic acids,
wherein each detection oligonucleotide comprises a different detectable label moiety; and
(iv)a detergent effective in preventing or disrupting non-specific interaction between each of said target nucleic acids and other components of said biological fluid;
(b) if any one of said target nucleic acids contains secondary, tertiary, or quaternary structure, heating the resulting mixture of step (a) to a temperature sufficient to disrupt said secondary, tertiary, or quaternary structure in said target nucleic acid;
(c) incubating said mixture to permit annealing of said capture and detection oligonucleotides to their respective target nucleic acid to form individual hybridization complexes between said target nucleic acids and their respective capture and detection oligonucleotides;
(d) binding each of said complexes to a substrate comprising the second member of said specific high affinity ligand pair of step (a)(ii) and removing any unbound material;
(e) determining the amount of each complex bound in step (d) by measuring the amount of said detectable label moiety or product thereof; and
(f) determining the concentration of each of said target nucleic acids in said biological fluid by comparing the amount of said detectable label moiety specific to each of said target nucleic acids with standard curves of each of said target nucleic acids determined in identical biological fluid or a representative surrogate matrix.

3. A method for determining the amount of a metabolite of a target nucleic acid in a biological fluid, comprising:
(a) mixing:
(i) a sample of biological fluid suspected of containing said metabolite of said target nucleic acid;
(ii) a capture oligonucleotide complementary to a first region of said metabolite of said target nucleic acid,
wherein said capture oligonucleotide comprises a first member of a specific high affinity ligand pair; and
(iii) a detection oligonucleotide complementary to a second region of said metabolite of said target nucleic acid,
wherein said detection oligonucleotide comprises a detectable label moiety; and
(iv) a detergent effective in preventing or disrupting non-specific interaction between said metabolite of said target nucleic acid and other components of said biological fluid;
(b) if said metabolite of said target nucleic acid contains secondary, tertiary, or quaternary structure, heating the resulting mixture of step (a) to a temperature sufficient to disrupt said secondary, tertiary, or quaternary structure;
(c) incubating said mixture to permit annealing of said capture and detection oligonucleotides to said metabolite of said target nucleic acid to form a hybridization complex between said metabolite of said target nucleic acid and said capture and detection oligonucleotides;
(d) binding said complex to a substrate comprising the second member of said specific high affinity ligand pair of step (a)(ii) and removing any unbound material;
(e) determining the amount of said complex bound in step (d) by measuring the amount of said detectable label moiety or product thereof; and
(f) determining the concentration of said metabolite of said target nucleic acid in said biological fluid by comparing the amount of said detectable label moiety with a standard curve of said metabolite determined in identical biological fluid or a representative surrogate matrix.

4. The method of claim 1, wherein said biological fluid is selected from the group consisting of blood, serum, saliva, urine, plasma, cerebrospinal fluid, follicular fluid, allantoic fluid, interstitial fluid, labyrinthine fluid, pericardial fluid, ventricular fluid, serous fluid, synovial fluid, tissue fluid, milk, seminal fluid, ocular fluid, amniotic fluid, placental fluid, ascitic fluid, pleural fluid, sputum, bronchial aspirate, and macerated tissue.

5. The method of claim 1, wherein the length of each of said capture and detection oligonucleotides is at least about 5 nucleotides.

6. The method of claim 1, wherein the members of said specific high affinity ligand pair are selected from the group consisting of biotin-streptavidin, an antibody-antigen, a receptor-ligand, an antibody-protein A, an antibody-protein G, a lectin-receptor, and a drug-receptor.

7. The method of claim 1, wherein said detectable label moiety is selected from the group consisting of a peptide, polypeptide, or protein; a fluorescent label; a bioluminescent label; a chemiluminescent label; a radioisotope; and a drug for which there exists a high affinity ligand.

8. The method of claim 1, wherein said peptide, polypeptide, or protein is detected by a method selected from the group consisting of reaction with an enzyme-linked antibody, gamma or scintillation counting, luminometry, phosphorescence, spectrophotometry, or spectrofluorometry.

9. The method of claim 1, wherein said enzyme-linked antibody is selected from the group consisting of an alkaline phosphatase-conjugated antibody, a horseradish peroxidase-conjugated antibody, a β-galactosidase-conjugated antibody, and a glucose oxidase-conjugated antibody.

10. The method of claim 1, wherein said detergent is selected from the group consisting of an anionic detergent, a cationic detergent, a nonionic detergent, and a zwitterionic detergent.

11. The method of claim 1, wherein said detergent is selected from the group consisting of sodium dodecyl sulfate, deoxycholic acid (3α,12α-dihydroxy-5β-cholan-24-oic acid), N-lauroyl-sarcosine, dodecyltrimethylammonium bromide, methylbenzethonium chloride (N,N-dimethyl-N-[2-(2-[methyl-4-(1,1,3,3-tetramethylbutyl)-phenoxy]-ethoxy)ethyl]benzylammonium chloride]; triton X-100 (t-octylphenoxypolyethoxyethanol), tween-20 (polyoxyethylenesorbitan monolaurate); and CHAPS (3-[(3-cholanimidopropyl)dimethyl-ammonio]-1-propane-sulfonate); and N-octyl-N-N-dimethyl-3-ammonio-1-propanesulfonate.

12. The method of claim 1, wherein said temperature sufficient to disrupt any secondary or higher structure in said target nucleic acid is in the range of from about 60° C. to about 100° C.

13. The method of claim 1, wherein said substrate is selected from the group consisting of a microtiter plate, a membrane, a filter, a glass bead, a plastic bead, a metal particle, a glass slide, a plastic slide, a glass tube, a plastic tube, a latex bead, and a latex sheet.

14. The method of claim 1, wherein said unbound material is removed by washing with a buffer, centrifugation, filtration, aspiration, decantation, or absorption.

15. The method of claim 1, wherein the amount of said detectable label is determined spectrophotometrically, spectrofluorometrically, by scintillation counting, gamma counting, by phosphorescence, by bioluminescence, or by chemiluminescence.

16. The method of claim 1, wherein said target nucleic acid is an oligonucleotide or a polynucleotide.

17. The method of claim 1, wherein said target nucleic acid is selected from the group consisting of a ribozyme; an antisense oligonucleotide; and a molecule comprising a nucleic acid covalently attached to a peptide, polypeptide, or protein; a non-peptide, polypeptide, or protein drug; a small organic molecule or drug; a sugar; or polyethylene glycol.

18. The method of claim 1, wherein said target nucleic acid is a therapeutically useful nucleic acid.

19. The method of claim 1, wherein said target nucleic acid is selected from the group consisting of an anti-hepatitis C virus ribozyme, an anti-hepatitis C virus antisense oligonucleotide, an anti-angiogenesis ribozyme, an anti-angiogenesis antisense oligonucleotide, an anti-HIV ribozyme, an anti-HIV antisense oligonucleotide, an anti-influenza ribozyme, an anti-influenza antisense oligonucleotide, an anti-rhinovirus ribozyme, and an anti-rhinovirus antisense oligonucleotide.

20. The method of claim 19, wherein said anti-hepatitis C virus ribozyme has the sequence shown in SEQ ID NO:1.

21. A method for determining the concentration of a ribozyme in mammalian serum, comprising:
(a) mixing:
(i) a sample of mammalian serum containing a ribozyme having the sequence shown in SEQ ID NO:1;
(ii) a capture oligonucleotide having the sequence shown in SEQ ID NO:2;
(iii) a detection oligonucleotide having the sequence shown in SEQ ID NO:3; and sodium dodecyl sulfate at a final concentration of 1% (w/v);
(b) heating the resulting mixture of step (a) to about 75° C. for about five minutes;
(c) incubating said mixture at 37° C. in 25 mM NaCl for two hours to form a hybridization complex between said target nucleic acid and said capture and detection oligonucleotides;
(d) binding said complex to a microtiter plate well comprising streptavidin and removing any unbound material;
(e) determining the amount of complex bound in step (d) by incubating said bound complex with an anti-digoxigenin alkaline phosphatase antibody and determining the corrected optical density of the resulting solution at 405 nm after addition of p-nitrophenyl phosphate; and
(f) determining the concentration of said ribozyme in said biological fluid from a standard curve of said ribozyme determined in the same manner as in (e) in otherwise identical control mammalian serum.

22. The method of claim 21, wherein said mammalian serum is selected from the group consisting of monkey serum, mouse serum, rat serum, and human serum.
